(19) United States
(12) Patent Application Publication
(10) Pub. No.: US 2021/0361469 A1
(43) Pub. Date: Nov. 25, 2021

(54) LIQUID ABSORBING BAG

(71) Applicants: Yi-Ching Liu, New Taipei City (TW); Hsin-Hua Tsai, Taipei City (TW)

(72) Inventors: Yi-Ching Liu, New Taipei City (TW); Hsin-Hua Tsai, Taipei City (TW)

(21) Appl. No.: 16/879,733

(22) Filed: May 20, 2020

(51) Int. Cl. A61F 5/453; A61F 5/44

(52) U.S. Cl. CPC A61F 5/453; A61F 5/4401

(57) ABSTRACT

A liquid absorbing bag adapted to be disposed on an excretory portion is provided, the excretory portion has an excretory outlet, and the liquid absorbing bag includes a liquid collecting bag and an absorbing pad. The liquid collecting bag defines a liquid accommodating space, and an opening communicating with the liquid accommodating space and allowing the excretory portion to enter the liquid accommodating space. The absorbing pad is arranged in the liquid accommodating space, and includes an absorbing sheet body. The absorbing sheet body has a folded portion disposed at an end of the liquid collecting bag away from the opening. The folded portion is folded along a plurality of substantially parallel fold lines and is expandable and compressible along a length direction perpendicular to the fold lines.

### BACKGROUND

### Technical Field

The invention relates to an absorbing bag, and particularly relates to a liquid absorbing bag.

### Description of Related Art

Male patients who stay in bed for a long time need others to regularly clean their bodies and deal with excrement. Normally, a caregiver may wrap an excretory portion with a diaper, so as to slightly alleviate the caregiver's burden. In order to avoid skin inflammation of the patient, even though the diaper has not absorbed much urine, the diaper should still be changed regularly. Therefore, the expense for diapers still causes a considerable cost of care.

On the other hand, since the conventional diapers are all in a planar shape, the caregiver needs to turn the patient over and wrap the diaper on the excretory portion at the same time during operation. The process is extremely time-consuming and laborious. In addition, since the absorbing area of the planar diaper cannot be fully utilized, the liquid absorbing efficiency is low.

### SUMMARY

The invention is directed to a liquid absorbing bag for the above issue.

The invention provides a liquid absorbing bag adapted to be disposed on an excretory portion, the excretory portion has an excretory outlet, and the liquid absorbing bag includes a liquid collecting bag and an absorbing pad. The liquid collecting bag defines a liquid accommodating space and an opening communicating with the liquid accommodating space and allowing the excretory portion to enter the liquid accommodating space. The absorbing pad is arranged in the liquid accommodating space, and includes an absorbing sheet body. The absorbing sheet body has a folded portion disposed at an end of the liquid collecting bag away from the opening. The folded portion is folded along a plurality of substantially parallel fold lines and is expandable and compressible along a length direction perpendicular to the fold lines.

In some embodiments, the absorbing sheet body further includes two extending end portions respectively extending from two opposite end edges of the folded portion in the length direction toward the opening along an inner surface of the liquid collecting bag.

In some embodiments, the liquid absorbing bag further includes two elastic bands, and the two elastic bands are respectively located on two opposite sides of the folded portion, and two opposite ends of each of the elastic bands are respectively connected to two opposite side surfaces of the liquid collecting bag in the length direction.

In some embodiments, the liquid collecting bag has an elastic surrounding portion adjacent to the opening, and the elastic surrounding portion is bound to a surface of the excretory portion through an elastic contraction force of the elastic surrounding portion.

In some embodiments, the absorbing sheet body has an absorbing layer, and a material of the absorbing layer includes cotton.

In some embodiments, the liquid collecting bag has a leak-proof layer, and a material of the leak-proof layer includes plastic.

In some embodiments, the liquid absorbing bag further includes a skin-friendly sheet body disposed on an inner surface of the liquid collecting bag and covering the absorbing sheet body. A material of the skin-friendly sheet body includes non-woven fabric.

In some embodiments, the liquid absorbing bag further includes two outer sheet bodies, which are respectively located on two opposite sides of the liquid collecting bag, and each of the outer sheet bodies is disposed on an outer surface of the liquid collecting bag, and the outer sheet body jointly defines an insertion space together with the liquid collecting bag for a finger to insert into the insertion space.

According to the embodiments of the invention, by compressing the folded portion in the length direction, most of the surface of the folded portion is located in an excretory area, thereby enlarging a surface capable of directly absorbing the liquid excrement, and improving liquid absorbing performance to keep the liquid accommodating space dry. Moreover, through the receiving spaces formed when the liquid collecting bag is compressed together, the caregiver may insert a single hand therein and apply a force to the folded portion, so that the folded portion is expanded in the length direction, and in the case that the folded portion enlarges the spacing between the extending end portions, the caregiver may approach the folded portion toward the excretory outlet. In general, with the folded portion being expandable and compressible, not only the time during which the liquid accommodating space is in the dry state is prolonged, but the assembling and disassembling of the liquid absorbing bag can also be more easily carried out by the caregiver.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic perspective view illustrating a liquid absorbing bag according to an embodiment and illustrates a position relationship between the liquid collecting bag and an absorbing pad of the embodiment.

FIG. 2 is a schematic cross-sectional view of FIG. 1 taken along a line II-II, and illustrates a detailed structure of the liquid collecting bag and the absorbing pad and that a folded portion is compressed in a length direction.

FIG. 3 is a schematic cross-sectional view according to another embodiment and illustrates that the folded portion is expanded in the length direction.

FIG. 4 is a schematic cross-sectional view of FIG. 1 according to another embodiment, and illustrates that a caregiver applies a force to two outer sheet bodies to expand the folded portion in the length direction.

### DESCRIPTION OF THE EMBODIMENTS

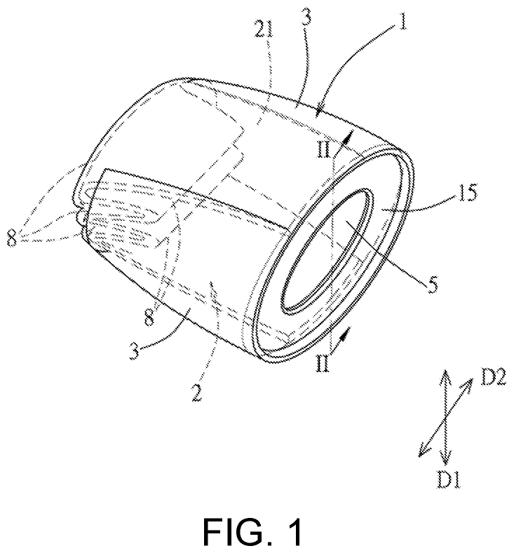

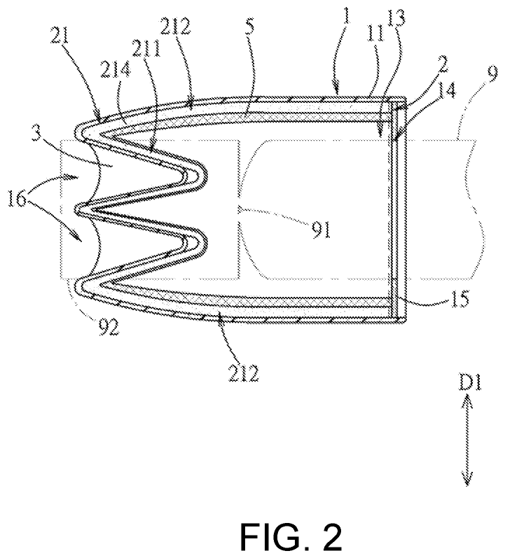

Referring to FIG. 1 and FIG. 2, a liquid absorbing bag of an embodiment of the invention is adapted to be sleeved on an excretory portion 9, the excretory portion 9 has an excretory outlet 91, and the liquid absorbing bag includes a liquid collecting bag 1, an absorbing pad 2, two elastic bands 3 and a skin-friendly sheet body 5. In the embodiment, the excretory portion 9 is, for example, a penis of a man, the liquid collecting bag 1 is adapted to be sleeved on the penis, and the absorbing pad 2 faces the excretory outlet 91 to receive liquid excrement (such as urine) discharged from the excretory outlet 91.

The liquid collecting bag 1 has a leak-proof layer 11. In the embodiment, a material of the leak-proof layer includes plastic. Based on a waterproof property of the plastic material, the liquid excrement overflowing from the absorbing pad 2 may be collected without overflowing outside the liquid collecting bag 1. However, the material of the leak-proof layer 11 is not limited to thereto.

The liquid collecting bag 1 defines a liquid accommodating space 13, and an opening 14 communicating with the liquid accommodating space 13 and allowing the excretory portion 9 to enter the liquid accommodating space 13. The liquid collecting bag 1 further has an elastic surrounding portion 15 adjacent to the opening 14, and the elastic surrounding portion 15 is bound to a surface of the excretory portion 9 through an elastic contraction force of the elastic surrounding portion 15. In the embodiment, the liquid accommodating space 13 is used for accommodating the excretory portion 9, and the elastic surrounding portion 15 extends inward radially from an inner surface of the liquid collecting bag 1 adjacent to the opening 14, and an inner peripheral edge of the elastic surrounding portion 15 leans against the surface of the excretory portion 9 tightly through the elastic contraction force, so that the overflowing liquid excrement can be kept in the liquid accommodating space 13 to restrict the liquid excrement from flowing to the outside through the opening 14. The elastic surrounding portion 15 is, for example, an elastic belt, but the type of the elastic surrounding portion 15 is not limited to the elastic belt.

The absorbing pad 2 is disposed in the liquid accommodating space 13, and includes an absorbing sheet body 21. In the embodiment, the absorbing pad 2 is installed on the inner surface of the liquid collecting bag 1 by adhesion, but the invention is not limited to adhesion.

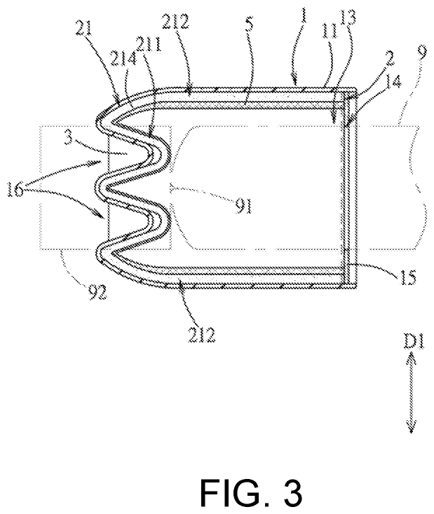

Referencing to FIG. 1, FIG. 2 and FIG. 3, the absorbing sheet body 21 has a folded portion 211 disposed at an end of the liquid collecting bag 1 away from the opening 14, and two extending end portions 212 respectively extending from two opposite end edges of the folded portion 211 toward the opening 14 along the inner surface of the liquid collecting bag 1. The folded portion 211 is folded along a plurality of substantially parallel fold lines 8 and is expandable (as shown in FIG. 3) or compressible (as shown in FIG. 2) along a length direction D1 perpendicular to the fold lines 8. In the embodiment, the folded portion 211 is folded along five parallel fold lines, and a cross-section thereof presents a reversed W-shape when the folded portion 211 is compressed in the length direction D1. Particularly, an excretory area 92 is defined to be located in front of the excretory outlet 91 and adjacent to the excretory outlet 91. In the embodiment, the folding structure of the folded portion 211 of the absorbing sheet body 21 can increase the proportion of the absorbing sheet body 21 in the excretory area 92, thereby facilitating liquid excrement absorption.

The portion of the liquid collecting bag 1 adhered to the folded portion 211 also presents a folded shape to form two receiving spaces 16 facing away from the opening 14. The receiving spaces 16 allow fingers of a caregiver to insert therein, and the caregiver may apply a force to the portion of the liquid collecting bag 1 fixed to the folded portion 211 by one hand, so that the folded portion 211 is expanded in the length direction D1. When the folded portion 211 is expanded in the length direction D1, a spacing between the extending end portions 212 is also increased, in this way, the caregiver may adjust the spacing to be greater than a width of the excretory portion 9 to more easily insert the excretory portion 9 between the extending end portions 212.

The extending end portions 212 respectively extend from two opposite end edges of the folded portion 211 in the length direction D1 toward the opening 14 along the inner surface of the liquid collecting bag 1, and is adapted to assist in absorbing the overflowing liquid excrement that is not absorbed by the folded portion 211, so as to keep the liquid accommodating space 13 dry.

The absorbing sheet body 21 has an absorbing layer 214. In the embodiment, the absorbing layer 214 is disposed on an inner surface of the leak-proof layer 11 and faces the opening 14. A material of the absorbing layer 214 includes cotton. The absorbing layer 214 may fully absorb the liquid excrement through cotton with the hygroscopic property, so as to keep the liquid accommodating space 13 dry. However, the material of the absorbing layer 214 is not limited to cotton. In another embodiment, the material of the absorbing layer 214 may also include non-woven fabric.

The elastic bands 3 are respectively located on two opposite sides of the folded portion 211 in a width direction D2 perpendicular to the length direction D1, and two opposite ends of each elastic band 3 are respectively connected to two opposite side surfaces of the liquid collecting bag 1 in the length direction D1. In the embodiment, each elastic band 3 extends in the length direction D1, with two opposite ends being respectively sewn on an outer surface of the liquid collecting bag 1 adjacent to the extending end portions 212. Through an elastic restoring force of the elastic band 3, the liquid collecting bag 1 and the extending end portions 212 become close to each other to keep the folded portion 211 in a compressed state. Certainly, the number of the elastic bands 3 is not limited thereto, and in another embodiment, the elastic bands 3 may also be adhered to surround the outer surface of the liquid collecting bag 1.

The skin-friendly sheet body 5 is sewn on the inner surface of the liquid collecting bag 1 and covers the absorbing sheet body 2. A material of the skin-friendly sheet body 5 includes non-woven fabric. With properties such as air permeability, softness, etc., of the non-woven fabric, the wearing comfort of the patient is improved. However, the material of the skin-friendly sheet body 5 is not limited thereto.

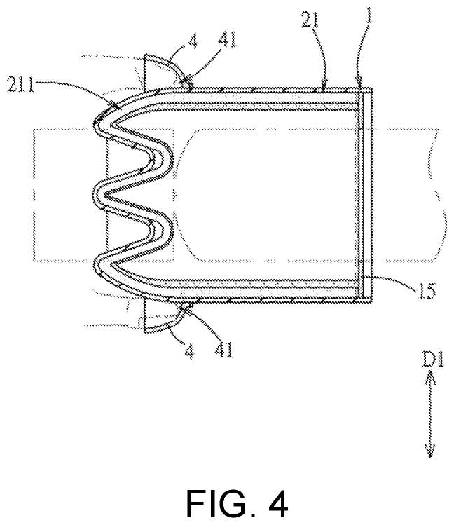

Moreover, referring to FIG. 4, in another implementation, the liquid absorbing bag further includes two outer sheet bodies 4, and the outer sheet bodies 4 are respectively located on two opposite sides of the liquid collecting bag 1, and each outer sheet body 4 is disposed on the outer surface of the liquid collecting bag 1, and jointly defines an insertion space 41 together with the liquid collecting bag 1 for a finger to insert into the insertion space 41. To be specific, the outer sheet bodies 4 are respectively sewn on the outer surface of the liquid collecting bag 1 corresponding to the two opposite ends of the absorbing sheet body 21, and define the insertion spaces 41 that open toward a left side of FIG. 4, so that a thumb and an index finger of the caregiver may respectively insert into the insertion spaces 41, and the caregiver may also apply a force to the outer sheet bodies 4 with one hand to drive the folded portion 211 to expand in the length direction D1.

In summary, based on the folding shape of the folded portion 211 of the liquid absorbing bag of the invention, the proportion of the absorbing sheet body 21 in the excretory area is increased, thereby enhancing the effect of absorbing the liquid excrement, and keeping the liquid accommodating space 13 dry. Moreover, the receiving spaces 16 formed by the liquid collecting bag 1 allow the caregiver to enlarge the spacing between the extending end portions 212 by one hand, thereby facilitating insertion of the excretory portion 9 between the extending end portions 212 to achieve the effect of the invention.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A liquid absorbing bag, adapted to be disposed on an excretory portion, the excretory portion having an excretory outlet, and the liquid absorbing bag comprising:
a liquid collecting bag, defining a liquid accommodating space and an opening communicating with the liquid accommodating space and allowing the excretory portion to enter the liquid accommodating space; and
an absorbing pad, arranged in the liquid accommodating space, and comprising an absorbing sheet body, wherein the absorbing sheet body has a folded portion disposed at an end of the liquid collecting bag away from the opening, and the folded portion is folded along a plurality of substantially parallel fold lines and is expandable and compressible along a length direction perpendicular to the fold lines.

2. The liquid absorbing bag as claimed in claim 1, wherein the absorbing sheet body further comprises two extending end portions respectively extending from two opposite end edges of the folded portion in the length direction toward the opening along an inner surface of the liquid collecting bag.

3. The liquid absorbing bag as claimed in claim 2, further comprising two elastic bands, the two elastic bands being respectively located on two opposite sides of the folded portion, and two opposite ends of each of the elastic bands being respectively connected to two opposite side surfaces of the liquid collecting bag in the length direction.

4. The liquid absorbing bag as claimed in claim 1, wherein the liquid collecting bag has an elastic surrounding portion adjacent to the opening, and the elastic surrounding portion is bound to a surface of the excretory portion through an elastic contraction force of the elastic surrounding portion.

5. The liquid absorbing bag as claimed in claim 1, wherein the absorbing sheet body has an absorbing layer, and a material of the absorbing layer comprises cotton.

6. The liquid absorbing bag as claimed in claim 1, wherein the liquid collecting bag has a leak-proof layer, and a material of the leak-proof layer comprises plastic.

7. The liquid absorbing bag as claimed in claim 1, further comprising a skin-friendly sheet body disposed on an inner surface of the liquid collecting bag and covering the absorbing sheet body, wherein a material of the skin-friendly sheet body comprises non-woven fabric.

8. The liquid absorbing bag as claimed in claim 1, further comprising two outer sheet bodies, respectively located on two opposite sides of the liquid collecting bag, wherein each of the outer sheet bodies is disposed on an outer surface of the liquid collecting bag, and the outer sheet body jointly defines an insertion space together with the liquid collecting bag for a finger to insert into the insertion space.
